(19) United States
(12) Patent Application Publication
(10) Pub. No.: US 2018/0008648 A1
(43) Pub. Date: Jan. 11, 2018

(54) SCAFFOLD-SEEDED ORAL MUCOSA STEM CELLS

(71) Applicants: Ramot at Tel-Aviv University Ltd., Tel-Aviv (IL); Technion Research & Development Foundation Limited, Haifa (IL)

(72) Inventors: Shulamit LEVENBERG, Moreshet (IL); Sandu PITARU, Ramat-Gan (IL); Daniel OFFEN, Kfar Haroeh (IL); Javier Ganz, Montevideo (UY); Sivan Ida COHEN-MATSLIAH, Atzmon-Segev (IL); Erez SHOR, Kiryat-Ono (IL)

(21) Appl. No.: 15/548,120

(22) PCT Filed: Jan. 31, 2016
(86) PCT No.: PCT/IL2016/050102

§ 371 Date: Aug. 2, 2017

Related U.S. Application Data
(60) Provisional application No. 62/110,639, filed on Feb. 2, 2015.

(51) Int. Cl. A61K 35/38; A61L 27/26; A61L 27/54; A61L 27/58; A61K 9/00; A61L 27/56; A61L 27/36

(52) U.S. Cl. CPC A61K 35/38; A61L 27/56; A61L 27/26; A61L 27/3604; A61L 27/58; A61K 9/0085; A61L 27/54; A61L 27/3675; A61L 2300/64; A61L 2430/32; A61L 2430/38

(57) ABSTRACT

A method of treating a spinal cord injury in a subject in need thereof is disclosed. The method comprises implanting a scaffold into the spinal cord of a subject, wherein the scaffold is seeded with oral mucosa stem cells (OMSC) and/or cells that have been ex vivo differentiated from said OMSCs, thereby treating the spinal cord injury.

### FIELD AND BACKGROUND OF THE INVENTION

The present invention, in some embodiments thereof, relates to methods of treating spinal cord injuries using scaffold-seeded oral mucosa stem cells and/or cells differentiated therefrom.

A goal of regenerative medicine is to regenerate the architecture and function of tissues and organs totally or partially lost due to disease, trauma and ageing. Stem cells are considered crucial building blocks for any regenerative strategy. The challenge and motivation are to find ways for recruiting and/or delivering to the injured site pluripotent stem cells populations capable of regenerating nonfunctional or lost tissues and organs. Bone marrow and to a very limited extent peripheral blood, fat, and muscle are the major sources for such a population. A serious drawback of these sources is that aging and disease substantially lower the functionality and possibly the availability of adult stem cells. Mesenchymal stem cells (MSCs) were suggested for regenerative therapy in the diseases involving neurodegeneration (Barzilay, R., Levy, Y. S., Isr Med Assoc J, 2006, 8, 61-66, Blondheim, N. R., et al., Stem Cells Dev., 2006, 15, 141-164; Sadan, O., Melamed, E. & Offen, D. Expert Opin Biol Ther., 2009, 9, 1487-1497).

Oral Mucosa is the mucosal lining the oral cavity, namely: the cheeks and the alveolar ridge including the gingiva and the palate, the tongue, the floor of the mouth and the oral part of the lips. Oral mucosa consists of an epithelial tissue of ectodermal origin and the lamina propria (LP) which is a connective tissue of ectomesenchymal origin. Similarly to the ectomesenchymal origin of connective tissues in the oral cavity, cells of the oral mucosa lamina propria (OMLP) originate from the embryonic ectodermal neural crest. Wounds in human oral mucosa heal mainly by regeneration.

The rate of healing is faster than that in the skin or other connective tissues and seems to be affected negligibly by age and gender (Szpaderska, A. M., et al., J Dent Res, 2003, 82, 621-626) Recently, the first evidence that the OMLP comprise a robust multipotent SC population with a neural crest-like stem cell phenotype was provided (PCT WO 2008/132722A1, EP 2 137 300 B1, Marynka-Kalmani, K., et al., Stem Cells, 2010, 28, 984-995). These findings positioned the human oral mucosa as a novel source for therapeutic adult SC. These authors also reported that explantation of the adult human OMLP reproducibly generates trillions of SC that they called, human oral mucosa stem cells (hOMSC). Immunophenotyping of hOMSC revealed a primitive neural crest stem cells (NCSC) phenotype, which is not affected by adult donor age.

The expression of pluripotency associated markers Oct4, Nanog and Sox2 and of the early neural crest stem/progenitor cell markers (Sox2 and p75) in vitro and in vivo points to the neural crest origin of this population and to the preservation of its primitiveness in the adult.

In vitro assays demonstrated that unsorted hOMSC subjected to neuronal differentiation regimens, differentiated into neuroectoderm lineages as evidenced by the decrease in Oct4 and Nanog, increase in MAP2 expression (neural) and the induction of neuritogenesis in PC12 cells, the last being considered a functional assay for glial differentiation (Bampton E T, Taylor J S. J Neurobiol 2005; 63:29-48).

Undifferentiated hOMSC however, supported only PC12 cells survival, probably via the secretion of nerve growth factor (NGF) and Fibroblast Growth Factor-2 (FGF-2). In addition, it was shown (Marynka-Kalmani, K., et al., ibid) that hOMSC can differentiate in vitro, into lineages of the three germ layers and after stimulation with dexamethasone, their implantation in vivo resulted in the formation of bilineage mixed tumors consisting of tissues that develop from cranial neural crest cells during embryogenesis. WO 2008/132722 discloses the lamina propria of the mucosa of the gastrointestinal tract and in particular of the oral mucosa, as a source for pluripotent adult stem cells.

U.S. Patent Application No. 20140335059 teaches use of oral mucosa stem cells for the treatment of neuronal disorders.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E: Characterization of hOMSC constructs. (A) Preparation scheme of naïve and induced hOMSC constructs. (B) Cell viability analysis of induced-constructs and naïve-constructs. Green indicates viable cells. Red indicates dead cells. Scale bar=500 um. (C) GFP signals after on-scaffold induction of hOMSCs. Scale bar (right)=60 um, scale bar (left), magnified region=10 um. (D) Upregulation of astrocyte markers GFAP (green) and EAAT1 (red). Induced-constructs (right) compared to naïve-constructs (left). Scale bar=100 um. (E) RT-PCR analysis of induced hOMSCs. Bars represent fold-increase compared to naïve hOMSCs. Comparison between 3D on-scaffold-induced hOMSCs (dotted bars) and hOMSCs induced in culture plates (solid bars): pluripotency and neural crest markers (green and magenta) neuronal markers (yellow astrocytic markers (red), and neurotrophic factors (blue).

FIGS. 2A-K: In-vivo analysis of therapeutic effects of implanted induced-constructs. (A) Implantation scheme. Following complete transection at T10, constructs seeded with cells or acellular scaffolds are implanted in the transection site and sealed with an acellular PLLA/PLGA scaffold. (B) Representative images of rat posture at experiment endpoint, following implantation of induced-construct (bottom) versus acellular scaffolds (top). (C) BBB scores over time of rats treated with induced-constructs (n=8, blue), naïve-constructs (n=9, green), OBC constructs (n=4, black) or acellular scaffolds (n=6, red). FM indicates the first measurement post-surgery, at days 1-4. (D) Coordinated gait analysis showing recovery of motor control in rats treated with induced-constructs. Gait pattern legend-hind-right (HR), front right (FR), hind left (HL), front left (FL). (E) Electrophysiology experiment design. The rat motor cortex was stimulated by single spikes. The contralateral sciatic nerve was exposed and MEPs were recorded. Following recording, the spinal cord was retransected at C5 and stimulation and recording were performed again to verify signal propagation through the spinal cord. (F) Representative recordings of the sciatic nerve in: intact rats (blue), rat treated with induced-constructs (ochre), rats treated with acellular scaffolds (black) and retransected rats treated with induced-constructs (green). (G) Quantification of MEP amplitudes of induced-constructs (n=3), acellular scaffold (n=3) and retransected induced-constructs (n=3). (H) Nociceptic perception test of the hind limbs and tail observed in rats treated with induced-constructs versus acellular scaffolds. (I) MRI-DTI fiber tracking (from left to right) on days 3 and 56, of rats treated with induced-constructs or acellular scaffold. Intact rats served as a reference. (J) 3D rendering of fibers overlaid on day 56-anatomical MRI data for rats treated with induced-constructs (right) versus rats treated with acellular scaffold (left). (K) FA analysis of intact rats, rats treated with induced-constructs (n=3) and rats treated with acellular scaffolds (n=3).

FIGS. 3A-B: Spinal cord immunofluorescence on day 56. (A) Immunofluorescence staining (from left to right—acellular constructs, naïve-constructs, induced-constructs) descending order, left panel A: Human nuclear staining, TUJ1 and NF200 co-localization, GAP43 expression, CSPGs, MBP and CD11b. Right panel A, descending order: GFAP, Nestin and spinal cord beta III tubulin image of induced-constructs and acellular constructs-treated animals. Scale bar=200 um, magnification insets scale bar=40um. (B) Computer-based quantification of staining. Top left—axonal and neuronal regeneration markers. Bottom left—inflammation, scarring and glial reactivity markers. Top right—neural precursor marker Nestin. Bottom left—quantification of MBP-expres sing elongated elements.

FIG. 4: Proteomic array analysis indicating expression fold-change of 80 proteins secreted by induced vs. naïve-constructs.

FIGS. 5A-B: Construct implantation procedure. The construct is shown implanted between the two transected spinal cord stumps (B). The sealing PLLA/PLGA scaffold is placed over the transection area and sutured in place (A).

FIG. 6: Fractional anisotropy (FA) maps of rats treated with an induced-construct (left) or acellular scaffold (right). Lighter colors represent higher FA values.

FIG. 7: H&E staining of the injury site. Top—acellular scaffold. Bottom induced-construct.

FIGS. 8A-B: Immunofluorescence image of GFP-labeled cells within the induced-construct, as observed at the end of the experiment.

FIG. 9 is an illustration of a single T-shaped scaffold according to embodiments described herein.

FIG. 10 is an illustration of two scaffolds which can make a T shape following implantation according to embodiments described herein.

FIG. 11A illustrates the positioning of an exemplary scaffold according to embodiments described herein following implantation.

FIG. 11B illustrates an exemplary penetrating scaffold according to embodiments described herein.

### DESCRIPTION OF SPECIFIC EMBODIMENTS OF THE INVENTION

The present invention, in some embodiments thereof, relates to methods of treating diseases using scaffold-seeded oral mucosa stem cells. According to a first aspect of the present invention there is provided a method of treating a spinal cord injury in a subject in need thereof comprising implanting a scaffold into the spinal cord of a subject, wherein the scaffold is seeded with oral mucosa stem cells (OMSC) and/or cells that have been ex vivo differentiated from said OMSCs, thereby treating the spinal cord injury.

As used herein, the phrase “spinal cord injury” refers to an injury to the spinal cord that is caused by trauma instead of disease. Depending on where the spinal cord and nerve roots are damaged, the symptoms can vary widely, for example from pain to paralysis to incontinence. Spinal cord injuries are described at various levels of “incomplete”, which can vary from having no effect on the patient to a “complete” injury which means a total loss of function. Spinal cord injuries have many causes, but are typically associated with major trauma from motor vehicle accidents, falls, sports injuries, and violence. The abbreviation “SCI” means spinal cord injury.

The spinal cord injury may be susceptible to secondary tissue injury, including but not limited to: glial scarring, myelin inhibition, demyelination, cell death, lack of neurotrophic support, ischemia, free-radical formation, and excitotoxicity.

Diseases of the spinal cord include but are not limited to autoimmune diseases (e.g. multiple sclerosis), inflammatory diseases (e.g. Arachnoiditis), neurodegenerative diseases, polio, spinabifida and spinal tumors.

The spinal cord injury may be an acute or chronic injury.

As used herein, the term “scaffold” refers to a three dimensional structure comprising a biocompatible material that provides a surface suitable for adherence and proliferation of cells. A scaffold may further provide mechanical stability and support.

It will be appreciated that the scaffold may be implanted as a single unit or as a plurality of units. When implanted as a single unit, the scaffold itself has a shape which comprises a T. Thus, the scaffold may be a T shaped scaffold or an H shaped scaffold. When implanted as a plurality of separate units, each individual unit may be of any shape (e.g. cylinders, blocks etc) as long as, after implantation they comprise a T shape.

It will be appreciated that the two arms of the T (i.e. the vertical arm and the horizontal arm) typically cross at right angles, although it will be appreciated that the angle may also be 99°, 98°, 97°, 96°, 55°, 94°, 93°, 92°, 91°, 89°, 88°, 87°, 86°, 85°, 84°, 83°, 82°, 81° or 80°.

In a preferred embodiment the horizontal arm of the T extends equally from both sides of the vertical arm.

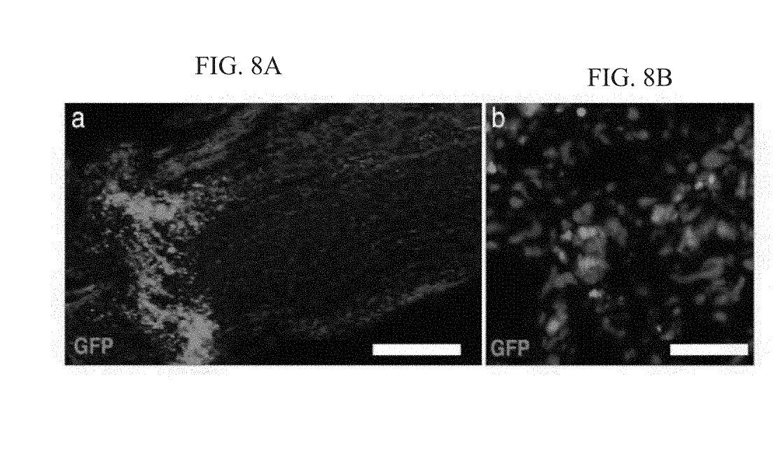

Referring now to FIG. 9, which illustrates a single scaffold having a T shape.

The horizontal section of the scaffold is referred to herein as the supporting section of the scaffold and the vertical section of the scaffold is referred to herein as the protruding section of the scaffold.

A thin, elongated cylinder is one possible configuration for the protruding section and/or horizontal section, but other shapes, such as elongated rectangular tubes, spheres, helical structures, and others are possible.

The dimensions of the scaffold will vary accordingly with the spinal cord lesion to be treated. For example, the length of the protruding section can be smaller than or substantially the same size as the depth of the lesion to be treated.

It will be further appreciated that the dimensions of the scaffold will vary according to the size of the subject. Thus, the dimensions of a scaffold for treating humans will be approximately ten or even twenty times greater than the dimensions of a scaffold for treating a small animal (e.g. rodent).

For a human, the height “d” of the protruding section, as illustrated in FIG. 9 is typically between 0.1 cm-3 cm, for example between 0.5 cm-3 cm, 0.5 cm-2 cm or 2-3 cm. For a rectangular protruding section, “e” may be between 0.1-2 cm, more preferably between 0.1-1 cm, more preferably between 0.1-0.5 cm and “f” may be between 0.1-2 cm, more preferably between 0.5-2 cm, more preferably between 0.5-1 cm. For a cylindrical protruding section, the diameter of the cylinder may be between 0.1-2 cm, more preferably between 0.5-2 cm, more preferably between 0.5-1 cm.

It will be appreciated that the protruding section may also be fashioned such that its shape mirrors the shape of the lesion to be treated.

The length of the supporting section “a” is typically between 2-10 cm, more preferably between 3-8 cm and even more preferably between 5-7 cm. The thickness “c” of the supporting section is typically between 0.5 cm-2 cm or 0.1 cm-1 cm.

According to one embodiment, the thickness “c” of the supporting section is greater than the thickness “f” of the protruding section. For example the ratio of c:f may be about 1.5: 1, 2:1, 3:1 or greater.

According to a preferred embodiment, the ratio a:e is greater than 2:1, 3:1, 4:1, 5: 1, 10:1 or even 20:1.

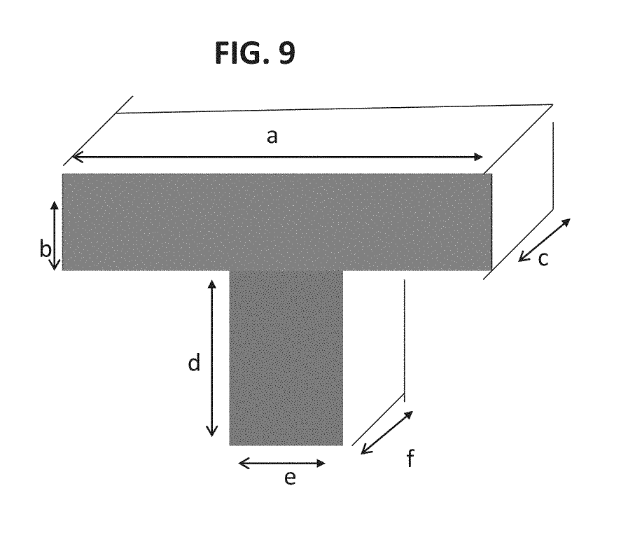

Referring now to FIG. 10, which illustrates two scaffolds which, following implantation, are capable of making a shape comprising a T shape. The scaffold which would be placed directly into the lesion is referred to herein as the protruding scaffold and is analogous to the protruding section of the scaffold described in FIG. 9 and the scaffold which would be placed on top of the protruding scaffold to generate the T shape is referred to herein as the supporting scaffold and is analogous to the supporting section of the scaffold described in FIG. 9.

A thin, elongated cylinder is one possible configuration for the protruding scaffold and/or horizontal scaffold, but other shapes, such as elongated rectangular tubes, spheres, helical structures, and others are possible.

The dimensions of the scaffolds will vary according to the spinal cord lesion to be treated. For example, the length of the protruding scaffold can be smaller than or substantially the same size as the depth of the lesion to be treated.

It will be further appreciated that the dimensions of the scaffolds will vary according to the size of the subject. Thus, the dimensions of scaffolds for treating humans will be approximately ten or even twenty times greater than the dimensions of scaffolds for treating a small animal (e.g. rodent).

For a human, the height “d” of the protruding scaffold, as illustrated in FIG. 9 is typically between 0.1 cm-3 cm, for example between 0.5 cm-3 cm, 0.5 cm-2 cm or 2-3 cm. For a rectangular protruding section, “e” may be between 0.1-2 cm, more preferably between 0.1-1 cm, more preferably between 0.1-0.5 cm and “f” may be between 0.1-2 cm, more preferably between 0.5-2 cm, more preferably between 0.5-1 cm. For a cylindrical protruding section, the diameter of the cylinder may be between 0.1-2 cm, more preferably between 0.5-2 cm, more preferably between 0.5-1 cm.

It will be appreciated that the protruding scaffold may also be fashioned such that its shape mirrors the shape of the lesion to be treated.

The length of the supporting scaffold “a” is typically between 2-10 cm, more preferably between 3-8 cm and even more preferably between 5-7 cm. The thickness “c” of the supporting scaffold is typically between 0.5 cm-2 cm or 0.1 cm-1 cm. According to one embodiment, the thickness “c” of the supporting scaffold is greater than the thickness “f” of the protruding scaffold. For example the ratio of c:f may be about 1.5: 1, 2:1, 3:1 or greater.

According to a preferred embodiment, the ratio a:e is greater than 2:1, 3:1, 4:1, 5: 1, 10:1 or even 20:1.

The scaffolds of the present invention may be made uniformly of a single polymer, co-polymer or blend thereof. However, it is also possible to form a scaffold according to the invention of a plurality of different polymers. There are no particular limitations to the number or arrangement of polymers used in forming the scaffold.

Any combination which is biocompatible, may be formed into fibers, and degrades at a suitable rate, may be used.

Both the choice of polymer and the ratio of polymers in a co-polymer may be adjusted to optimize the stiffness of the scaffold. The molecular weight and cross-link density of the scaffold may also be regulated to control both the mechanical properties of the scaffold and the degradation rate (for degradable scaffolds). The mechanical properties may also be optimized to mimic those of the tissue at the implant site.

Scaffold material may comprise natural or synthetic organic polymers that can be gelled, or polymerized or solidified (e.g., by aggregation, coagulation, hydrophobic interactions, or cross-linking) into a 3-D open-lattice structure that entraps water or other molecules, e.g., to form a hydrogel. Structural scaffold materials may comprise a single polymer or a mixture of two or more polymers in a single composition.

Additionally, two or more structural scaffold materials may be co-deposited so as to form a polymeric mixture at the site of deposition. Polymers used in scaffold material compositions may be biocompatible, biodegradable and/or bioerodible and may act as adhesive substrates for cells. In exemplary embodiments, structural scaffold materials are easy to process into complex shapes and have a rigidity and mechanical strength suitable to maintain the desired shape under in vivo conditions.

In certain embodiments, the structural scaffold materials may be non-resorbing or non-biodegradable polymers or materials.

The phrase “non-biodegradable polymer”, as used herein, refers to a polymer or polymers which at least substantially (i.e. more than 50%) do not degrade or erode in vivo. The terms “non-biodegradable” and “non-resorbing” are equivalent and are used interchangeably herein.

Such non-resorbing scaffold materials may be used to fabricate materials which are designed for long term or permanent implantation into a host organism. In exemplary embodiments, non-biodegradable structural scaffold materials may be biocompatible. Examples of biocompatible non-biodegradable polymers which are useful as scaffold materials include, but are not limited to, polyethylenes, polyvinyl chlorides, polyamides such as nylons, polyesters, rayons, polypropylenes, polyacrylonitriles, acrylics, polyisoprenes, polybutadienes and polybutadiene-polyisoprene copolymers, neoprenes and nitrile rubbers, polyisobutylenes, olefinic rubbers such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates such as ethylene vinyl acetate copolymer, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers such as polytetrafluoroethylene and polyvinyl fluoride, polystyrenes, homopolymers and copolymers of styrene acrylonitrile, cellulose acetates, homopolymers and copolymers of acrylonitrile butadiene styrene, polymethylpentenes, polysulfones, polyesters, polyimides, polyisobutylenes, polymethylstyrenes, and other similar compounds known to those skilled in the art.

In other embodiments, the structural scaffold materials may be a “bioerodible” or “biodegradable” polymer or material.

The phrase “biodegradable polymer” as used herein, refers to a polymer or polymers which degrade in vivo, and wherein erosion of the polymer or polymers over time occurs concurrent with or subsequent to release of the islets. The terms “biodegradable” and “bioerodible” are equivalent and are used interchangeably herein.

Such bioerodible or biodegradable scaffold materials may be used to fabricate temporary structures. In exemplary embodiments, biodegradable or bioerodible structural scaffold materials may be biocompatible. Examples of biocompatible biodegradable polymers which are useful as scaffold materials include, but are not limited to, polylactic acid, polyglycolic acid, polycaprolactone, and copolymers thereof, polyesters such as polyglycolides, polyanhydrides, polyacrylates, polyalkyl cyanoacrylates such as n-butyl cyanoacrylate and isopropyl cyanoacrylate, polyacrylamides, polyorthoesters, polyphosphazenes, polypeptides, polyurethanes, polystyrenes, polystyrene sulfonic acid, polystyrene carboxylic acid, polyalkylene oxides, alginates, agaroses, dextrins, dextrans, polyanhydrides, biopolymers such as collagens and elastin, alginates, chitosans, glycosaminoglycans, and mixtures of such polymers. In still other embodiments, a mixture of non-biodegradable and bioerodible and/or biodegradable scaffold materials may be used to form a biomimetic structure of which part is permanent and part is temporary.

PLA, PGA and PLA/PGA copolymers are particularly useful for forming the scaffolds of the present invention. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. The erosion of the polyester scaffold is related to the molecular weights. The higher molecular weights, weight average molecular weights of 90,000 or higher, result in polymer scaffolds which retain their structural integrity for longer periods of time; while lower molecular weights, weight average molecular weights of 30,000 or less, result in both slower release and shorter scaffold lives. For example, poly(lactide-co-glycolide) (50:50) degrades in about six weeks following implantation.

According to a preferred embodiment of this aspect of the present invention the scaffold comprises a 50:50 blend of (1) poly(lactic-co-glycolic acid) and (2) poly-L-lactic acid (PLLA). It is preferred that any of the foregoing articles have a degradation rate of about between about 30 and 90 days (e.g. about 6 weeks, 7 weeks, eight weeks, nine week or ten weeks); however, the rate can be altered to provide a desired level of efficacy of treatment.

The molecular weight (MW) of the polymers used to fabricate the presently described scaffolds can vary according to the polymers used and the degradation rate desired to be achieved. In one embodiment, the average MW of the polymers in the scaffold is between about 1,000 and about 50,000. In another embodiment, the average MW of the polymers in the scaffold is between about 2,000 and 30,000. In yet another embodiment, the average MW is between about 20,000 and 50,000 for PLGA and between about 300,000 and 500,000 for PLLA.

Advantageously, the polymeric material may be fabricated as a putty. By “putty” it is meant that the material has a dough-like consistency that is formable or moldable. These materials are sufficiently and readily moldable such that they can be carved into flexible three-dimensional structures or shapes complementary to a target site to be treated.

In certain embodiments, the structural scaffold material composition is solidified or set upon exposure to a certain temperature; by interaction with ions, e.g., copper, calcium, aluminum, magnesium, strontium, barium, tin, and di-, tri- or tetra-functional organic cations, low molecular weight dicarboxylate ions, sulfate ions, and carbonate ions; upon a change in pH; or upon exposure to radiation, e.g., ultraviolet or visible light. In an exemplary embodiment, the structural scaffold material is set or solidified upon exposure to the body temperature of a mammal, e.g., a human being. The scaffold material composition can be further stabilized by cross-linking with a polyion.

In an exemplary embodiment, scaffold materials may comprise naturally occurring substances, such as, fibrinogen, fibrin, thrombin, chitosan, collagen, alginate, poly(N-isopropylacrylamide), hyaluronate, albumin, synthetic polyamino acids, prolamines, polysaccharides such as alginate, heparin, and other naturally occurring biodegradable polymers of sugar units.

In certain embodiments, structural scaffold materials may be ionic hydrogels, for example, ionic polysaccharides, such as alginates or chitosan. Ionic hydrogels may be produced by cross-linking the anionic salt of alginic acid, a carbohydrate polymer isolated from seaweed, with ions, such as calcium cations. The strength of the hydrogel increases with either increasing concentrations of calcium ions or alginate. For example,

U.S. Pat. No. 4,352,883 describes the ionic cross-linking of alginate with divalent cations, in water, at room temperature, to form a hydrogel matrix. In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups.

Examples of polymers with basic side groups that can react with anions are poly(vinyl amines), poly(vinyl pyridine), and poly(vinyl imidazole). Polyphosphazenes are polymers with backbones consisting of nitrogen and phosphorous atoms separated by alternating single and double bonds. Each phosphorous atom is covalently bonded to two side chains. Polyphosphazenes that can be used have a majority of side chains that are acidic and capable of forming salt bridges with di- or trivalent cations. Examples of acidic side chains are carboxylic acid groups and sulfonic acid groups. Bioerodible polyphosphazenes have at least two differing types of side chains, acidic side groups capable of forming salt bridges with multivalent cations, and side groups that hydrolyze under in vivo conditions, e.g., imidazole groups, amino acid esters, glycerol, and glucosyl. Bioerodible or biodegradable polymers, i.e., polymers that dissolve or degrade within a period that is acceptable in the desired application (usually in vivo therapy), will degrade in less than about five years or in less than about one year, once exposed to a physiological solution of pH 6-8 having a temperature of between about 25° C. and 38° C. Hydrolysis of the side chain results in erosion of the polymer. Examples of hydrolyzing side chains are unsubstituted and substituted imidizoles and amino acid esters in which the side chain is bonded to the phosphorous atom through an amino linkage.

Typically, the scaffolds of the present invention are porous. The porosity of the scaffold may be controlled by a variety of techniques known to those skilled in the art. The minimum pore size and degree of porosity is dictated by the need to provide enough room for the cells and for nutrients to filter through the scaffold to the cells.

The maximum pore size and porosity is limited by the ability of the scaffold to maintain its mechanical stability after seeding. As the porosity is increased, use of polymers having a higher modulus, addition of stiffer polymers as a co-polymer or mixture, or an increase in the cross-link density of the polymer may all be used to increase the stability of the scaffold with respect to cellular contraction.

According to a preferred embodiment of this aspect of the present invention, the scaffold has an average pore diameter of about 100-1000 μm, more preferably between 300-600 μm and even more preferably between 400-500 μm.

Electrical signals in the form of action potentials are the means of signaling for billions of cells in the central nervous system. Numerous studies have shown that this electrical activity is not only a means of communication, but also necessary for the normal development of the nervous system and refinement of functional neural circuits.

In the case of spinal cord injury, cell-to-cell communication may be interrupted and the mechanisms of normal neurological development imply that electrical activity should be part of the restoration of functional connections. Such activity is important for the survival of existing cells and the incorporation of any transplanted cells into working circuits. In an embodiment of the present invention, the scaffolds are fabricated from synthetic biomaterials and are capable of conducting electricity and naturally eroding inside the body. In an exemplary embodiment, the scaffolds comprise a biocompatible polymer capable of conducting electricity e.g. a polypyrrole polymer. Polyaniline, polyacetyline, poly-p-phenylene, poly-p-phenylene-vinylene, polythiophene, and hemosin are examples of other biocompatible polymers that are capable of conducting electricity and may be used in conjunction with the present invention. Other erodible, conducting polymers are well known (for example, see Zelikin et al., Erodible Conducting Polymers for Potential Biomedical Applications, Angew. Chem. Int. Ed. Engl., 2002, 41(1):141-144). Any of the foregoing electrical conducting polymers can be applied or coated onto a malleable or moldable scaffold.

The scaffolds may be made by any of a variety of techniques known to those skilled in the art. Salt-leaching, porogens, solid-liquid phase separation (sometimes termed freeze-drying), and phase inversion fabrication may all be used to produce porous scaffolds. Fiber pulling and weaving (see, e.g. Vacanti, et al., (1988) Journal of Pediatric Surgery, 23: 3-9) may be used to produce scaffolds having more aligned polymer threads. Those skilled in the art will recognize that standard polymer processing techniques may be exploited to create polymer scaffolds having a variety of porosities and microstructures.

Scaffold materials are readily available to one of ordinary skill in the art, usually in the form of a solution (suppliers are, for example, BDH, United Kingdom, and Pronova Biomedical Technology a.s. Norway). For a general overview of the selection and preparation of scaffolding materials, see the American National Standards Institute publication No. F2064-00 entitled Standard Guide for Characterization and Testing of Alginates as Starting Materials Intended for Use in Biomedical and Tissue Engineering Medical Products Applications”.

Therapeutic compounds or agents that modify cellular activity can also be incorporated (e.g. attached to, coated on, embedded or impregnated) into the scaffold material. Campbell et al. (US Patent Application No. 20030125410) which is incorporated by reference as if fully set forth by reference herein, discloses methods for fabrication of 3D scaffolds for stem cell growth, the scaffolds having preformed gradients of therapeutic compounds. The scaffold materials, according to Campbell et al, fall within the category of “bio-inks”. Such “bio-inks” are suitable for use with the compositions and methods of the present invention.

Exemplary agents that may be incorporated into the scaffold of the present invention include, but are not limited to those that promote cell adhesion (e.g. fibronectin, integrins), cell colonization, cell proliferation, cell differentiation, anti-inflammatories, cell extravasation and/or cell migration. Thus, for example, the agent may be an amino acid, a small molecule chemical, a peptide, a polypeptide, a protein, a DNA, an RNA, a lipid and/or a proteoglycan.

Proteins that may be incorporated into the scaffolds of the present invention include, but are not limited to extracellular matrix proteins, cell adhesion proteins, growth factors, cytokines, hormones, proteases and protease substrates. Thus, exemplary proteins include vascular endothelial-derived growth factor (VEGF), activin-A, retinoic acid, epidermal growth factor, bone morphogenetic protein, TGFβ, hepatocyte growth factor, platelet-derived growth factor, TGFα, IGF-I and II, hematopoetic growth factors, heparin binding growth factor, peptide growth factors, erythropoietin, interleukins, tumor necrosis factors, interferons, colony stimulating factors, basic and acidic fibroblast growth factors, nerve growth factor (NGF) or muscle morphogenic factor (MMP). The particular growth factor employed should be appropriate to the desired cell activity. The regulatory effects of a large family of growth factors are well known to those skilled in the art.

The protruding scaffold (and optionally the supporting scaffold) is typically seeded with cells prior to implantation. The cells in the protruding scaffold and supporting scaffold may be identical or non-identical. Due to the size of the supporting scaffold, typically the ratio of the number of cells in the supporting scaffold is greater than 2:1, 3:1 or even 4:1.

As mentioned, the stem cells used in this aspect of the present invention are stem cells derived from the oral mucosa (or are ex vivo differentiated from said stem cells). The term “oral mucosa” refers to the mucosal lining the oral cavity, namely: the cheeks and the alveolar ridge including the gingiva and the palate, the tongue, the floor of the mouth and the oral part of the lips.

Oral mucosa stem cells (OMSCs) have been described in U.S. Patent Application No. 20140335059, the contents of which are incorporated herein by reference. Human OMSC express general neuronal markers constitutively, such as TUJ1 and MAP2. In one embodiment, the OMSC express dopaminergic markers NURR1, LMX1A and low levels of TH, phenomena.

Separation of the stem cells according to the present invention may be performed according to various physical properties, such as fluorescent properties or other optical properties, magnetic properties, density, electrical properties, etc. Cell types can be isolated by a variety of means including fluorescence activated cell sorting (FACS), protein-conjugated magnetic bead separation, morphologic criteria, specific gene expression patterns (using RT-PCR), or specific antibody staining.

The use of separation techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye Hoechst 33342).

Cells may be selected based on light-scatter properties as well as their expression of various cell surface antigens. The purified stem cells have low side scatter and low to medium forward scatter profiles by FACS analysis. Cytospin preparations show the enriched stem cells to have a size between mature lymphoid cells and mature granulocytes.

Various techniques can be employed to separate the cells by initially removing cells of dedicated lineage. Monoclonal antibodies are particularly useful. The antibodies can be attached to a solid support to allow for crude separation. The separation techniques employed should maximize the retention of viability of the fraction to be collected.

The separation techniques employed should maximize the retention of viability of the fraction to be collected. Various techniques of different efficacy may be employed to obtain “relatively crude” separations. Such separations are where up to 30%, usually not more than about 5%, preferably not more than about 1%, of the total cells present are undesired cells that remain with the cell population to be retained.

The particular technique employed will depend upon efficiency of separation, associated cytotoxicity, ease and speed of performance, and necessity for sophisticated equipment and/or technical skill.

Procedures for separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., plate, or other convenient technique.

Techniques providing accurate separation include fluorescence activated cell sorters, which can have varying degrees of sophistication, e.g., a plurality of color channels, low angle and obtuse light scattering detecting channels, impedance channels, etc.

Other techniques for positive selection may be employed, which permit accurate separation, such as affinity columns, and the like.

Antibodies used for separation may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the remaining cells.

While it is believed that the particular order of separation is not critical to this invention, the order indicated is preferred. Preferably, cells are initially separated by a coarse separation, followed by a fine separation, with positive selection of one or more markers associated with the stem cells and negative selection for markers associated with lineage committed cells.

The freezing of cells is ordinarily destructive. On cooling, water within the cell freezes. Injury then occurs by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroys the cell. These injurious effects can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, and inorganic salts.

In a preferred embodiment, DMSO is used, a liquid which is nontoxic to cells in low concentrations. DMSO freely permeates the cell and serves as a cryoprotectant.

Cryoprotectants protect intracellular organelles by combining with water to modify its freezability and prevent damage from ice formation. Addition of plasma (e.g., to a concentration of 20-25%) can augment the protective effect of DMSO. After addition of DMSO, cells should be kept at 0° C. until freezing, since DMSO concentrations of about 1% are toxic at temperatures above 4° C.

A controlled slow cooling rate is critical. Different cryoprotective agents and different cell types have different optimal cooling rates (Lewis, J. P., et al. Transfusion 7, 17-32, 1967). The heat of fusion phase where water turns to ice should be minimal.

The cooling procedure can be carried out by use of, e.g., a programmable freezing device or a methanol bath procedure. Programmable freezing apparatuses allow determination of optimal cooling rates and facilitate standard reproducible cooling. Programmable controlled-rate freezers such as Cryomed or Planar permit tuning of the freezing regimen to the desired cooling rate curve. For example, for marrow cells in 10% DMSO and 20% plasma, the optimal rate is 1 to 3° C./minute from 0° C. to −80 ° C. In a preferred embodiment, this cooling rate can be used for the cells of the invention. The container holding the cells must be stable at cryogenic temperatures and allow for rapid heat transfer for effective control of both freezing and thawing. Sealed plastic vials (e.g., Nunc, Wheaton cryules) or glass ampoules can be used for multiple small amounts (1-2 ml), while larger volumes (100-200 ml) can be frozen in polyolefin bags (e.g., Delmed) held between metal plates for better heat transfer during cooling. (Bags of bone marrow cells have been successfully frozen by placing them in −80 ° C. freezers which, fortuitously, gives a cooling rate of approximately 3° C./minute).

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred aspect, DMSO-treated cells are pre-cooled on ice and transferred to a tray containing chilled methanol which is placed, in turn, in a mechanical refrigerator (e.g., Harris or Revco) at −80° C.

Thermocouple measurements of the methanol bath and the samples indicate the desired cooling rate of 1 to 3° C./minute. After at least two hours, the specimens have—reached a temperature of −8° C. and can be placed directly into liquid nitrogen (−196° C. for permanent storage.

After thorough freezing, cells can be rapidly transferred to a long-term cryogenic storage vessel. In a preferred embodiment, samples can be cryogenically stored in liquid nitrogen (−196° C. or its vapor (−165° C.). Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

Methods of cryopreservation of viable cells, or modifications thereof, are available and envisioned for use (e.g., cold metal-mirror techniques; U.S. Pat. No. 4,199,022; U.S. Pat. No. 3,753,357; U.S. Pat. No. 4,559,298). U.S. Pat. No. 6,310,195 discloses a method for preservation of pluripotent progenitor cells, as well as totipotent progenitor cells based on a use of a specific protein. In a preferred case, the protein can preserve hematopoietic progenitor cells, but progenitor cells from other tissues can also be preserved, including nerve, muscle, skin, gut, bone, kidney, liver, pancreas, or thymus progenitor cells.

Frozen cells are preferably thawed quickly (e.g., in a water bath maintained at 37-41° C.) and chilled immediately upon thawing. In particular, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In Vitro Culture and Expansion of Stem Cells: An optional procedure (either before or after cryopreservation) is to expand the stem in vitro. However, care should be taken to ensure that growth in vitro does not result in the production of differentiated progeny cells at the expense of multipotent stem cells which are therapeutically necessary for reconstitution.

Stem cells contained in the oral mucosa may be differentiated, using specific protocols, into dopaminergic or astrocyte neural cells and used for prevention and treatment of neurodegenerative diseases and disorders. In addition, whole populations of oral mucosa can be used without requiring laborious purification, as a source for multipotent stem cells capable of differentiating into neural cell lineages under in vivo and/or in vitro conditions.

An exemplary method of ex vivo differentiating oral mucosa stem cells into neurotrophic factor releasing cells is provided herein below:

Neuron Supporting Cell Induction of hOMSC: A two-step medium based differentiation protocol may be performed. In the first step, the cells are incubated in serum free conditions (DMEM low glucose/SPN/Glutamine) with the addition of N2 supplement (GIBCO), basic Fibroblast Growth Factor 2 (bFGF) (R&D Systems) and Epidermal Growth Factor (EGF) (R&D Systems) at a 20 ng/mL final concentration.

Following 72 hr, the second differentiation step is initiated. Cells are incubated in serum free medium (DMEM low glucose/SPN/Glutamine) with the addition of dbcAMP (1 mM) (SIGMA), IBMX (0.5 mM) (SIGMA), Neuregulin (50 ng/mL) and PDGF (1 ng/mL) (Peprotech) for additional 72 hrs. The differentiation protocol may be performed in cells that didn't undergo more than ten passages.

The cells may be genetically modified or non-genetically modified.

According to a particular embodiment, the cells are human.

According to a particular embodiment, a portion of the penetrating scaffold is seeded with cells and a portion of the penetrating scaffold is not seeded with cells.

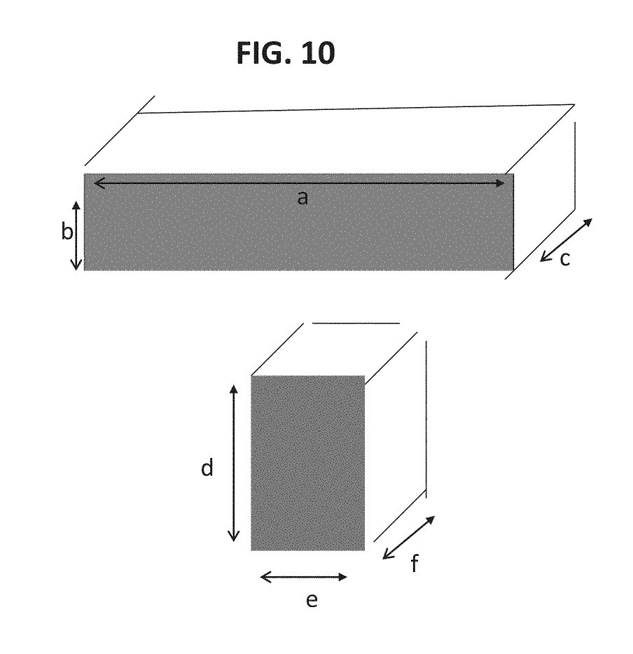

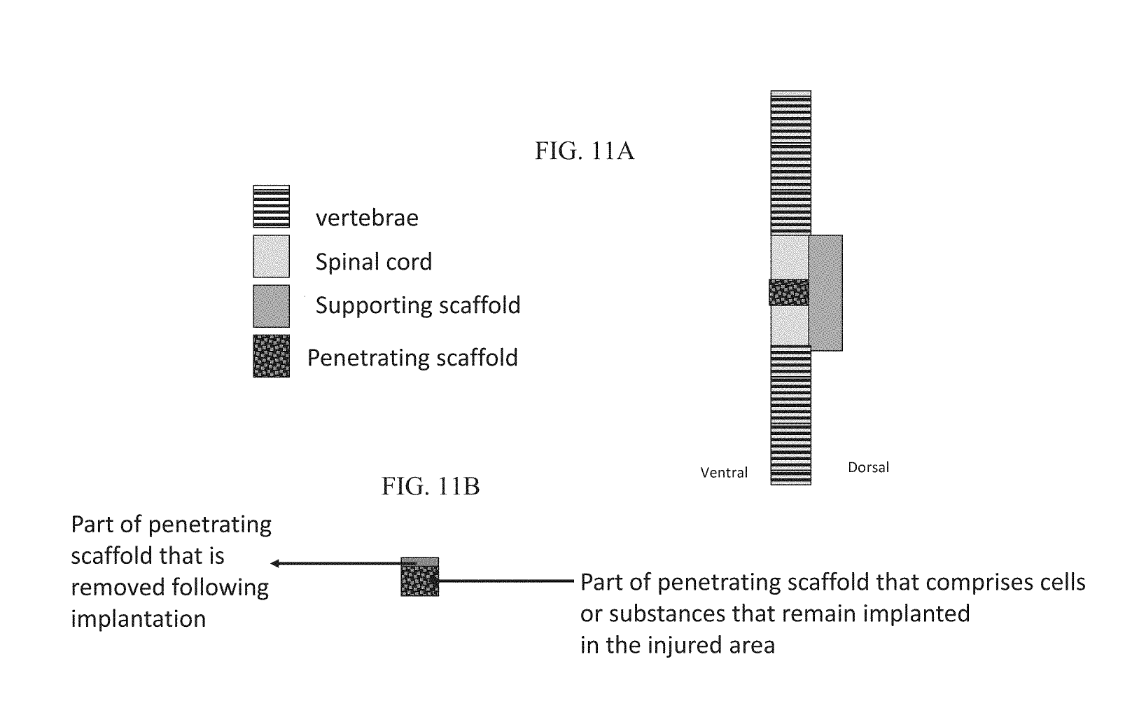

The portion of the scaffold which is not seeded with cells is typically the part of the scaffold that is in contact with the implantation device (e.g. tweezers) during the implantation procedure (as illustrated in FIG. 11B). This portion of the scaffold may be removed following implantation.

Cells can be seeded in the scaffold by static loading, or, more preferably, by seeding in stirred flask bioreactors (scaffold is typically suspended from a solid support), in a rotating wall vessel, or using direct perfusion of the cells in medium in a bioreactor. Highest cell density throughout the scaffold is achieved by the latter (direct perfusion) technique.

The cells may be seeded directly onto the scaffold, or alternatively, the cells may be mixed with a gel which is then absorbed onto the interior and exterior surfaces of the scaffold and which may fill some of the pores of the scaffold. Capillary forces will retain the gel on the scaffold before hardening, or the gel may be allowed to harden on the scaffold to become more self-supporting. Alternatively, the cells may be combined with a cell support substrate in the form of a gel optionally including extracellular matrix components. An exemplary gel is Matrigel™, from Becton-Dickinson. Matrigel™ is a solubilized basement membrane matrix extracted from the EHS mouse tumor (Kleinman, H. K., et al., Biochem. 25:312, 1986). The primary components of the matrix are laminin, collagen I, entactin, and heparan sulfate proteoglycan (perlecan) (Vukicevic, S., et al., Exp. Cell Res. 202:1, 1992). Matrigel™ also contains growth factors, matrix metalloproteinases (MMPs [collagenases]), and other proteinases (plasminogen activators [PAs]) (Mackay, A. R., et al., BioTechniques 15:1048, 1993). The matrix also includes several undefined compounds (Kleinman, H. K., et al., Biochem. 25:312, 1986; McGuire, P. G. and Seeds, N. W., J. Cell. Biochem. 40:215, 1989), but it does not contain any detectable levels of tissue inhibitors of metalloproteinases (TIMPs) (Mackay, A. R., et al., BioTechniques 15:1048, 1993). Alternatively, the gel may be growth-factor reduced Matrigel, produced by removing most of the growth factors from the gel (see Taub, et al., Proc. Natl. Acad. Sci. USA (1990); 87 (10:4002-6). In another embodiment, the gel may be a collagen I gel, alginate, or agar. Such a gel may also include other extracellular matrix components, such as glycosaminoglycans, fibrin, fibronectin, proteoglycans, and glycoproteins. The gel may also include basement membrane components such as collagen IV and laminin. Enzymes such as proteinases and collagenases may be added to the gel, as may cell response modifiers such as growth factors and chemotactic agents.

According to a particular embodiment, the gel comprises fibrin.

For treating spinal cord injuries (e.g. a compression spinal cord injury), the protruding scaffold (or protruding section of the single scaffold) is implanted directly into the wound (e.g. into the epicenter of the injury), wherein the scaffold runs through the injury site as illustrated in FIG. 11A. The scaffold can be inserted through a surgical incision directly into the lesion to be treated.

Following implantation of the protruding scaffold, the supporting scaffold is implanted. The supporting scaffold extends beyond the caudal and rostral sides of the injured site and preferably at a distance of approximately ¼ or ½ the length of the injured site. In a preferred embodiment supporting scaffold will extend equally beyond the caudal and rostral sides of the injured.

The supporting scaffold does not protrude into the injury or diseased site and is in contact with the rostral and/or caudal dura of the spinal cord. Further, the supporting scaffold is implanted such that it is in direct contact with the penetrating scaffold—see FIG. 11A. Following implantation of the supporting scaffold, the muscle layer above is sutured such that it presses against the area of the spinal cord and greatly reduces the movement of the spinal cord. By constraining the spinal cord in this way, and reducing movement, glial scar formation is reduced.

It is expected that during the life of a patent maturing from this application many relevant scaffolds will be developed and the scope of the term scaffold is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical of a condition or substantially preventing the appearance of clinical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed.

(1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Spinal cord injury, involving damaged axons and glial scar, often culminates in irreversible impairments. Achieving substantial recovery following complete spinal cord transection is still an unmet challenge. Here, we report of implantation of a 3D construct bearing human oral mucosa stem cells (hOMSC) induced to secrete neuroprotective, immunomodulatory and regeneration-associated factors in a complete spinal cord transection rat model. At three weeks postimplantation, 63% of the hOMSC-treated rats regained locomotor abilities, coordination and nociceptic perception, and displayed Basso-Beattie-Bresnahan (BBB) locomotor scores 17, while 83% of the animals treated with acellular constructs remained paralyzed, with BBB scores 2. Imaging and electrophysiology confirmed a functional reconnection bridging the caudal and rostral parts of the injured area. An increased number of myelinated axons, reduced glial scar and an augmented number of neural precursors were histologically observed. Thus, hOMSC-embedded constructs maintain therapeutic cells in the lesion site and elicit substantial functional recovery after spinal injury.

Spinal cord injury (SCI) results in structural and functional damage to neural circuitry due to axon damage, intense local inflammation, glial scarring and progressive tissue cavitation that extends beyond the boundaries of the primary lesion¹. Experimental allograft nerve transplantation, cell therapies and tissue engineering have led to partial functional recovery in rodents²⁴. A recent work in humans demonstrated the beneficial effects of transplanted autologous bulbar cells⁶. hOMSC derived from the lamina propria of the readily accessible oral mucosa⁶ are compelling candidates for cell therapy. hOMSCs exhibit a neural crest-like stem cell phenotype, high and stable expandability, with a capacity of over 70 cumulative population doublings, low interdonor heterogeneity and a negligible effect of aging on clonogenicity, growth and differentiation potential⁶. In addition, these cells secrete a variety of growth factors known to induce regenerative and neuroprotective processes. Moreover, we recently reported that hOMSCs can be induced into GFAP- and S100β-positive astrocyte-like cells, which secreted increased levels of neurotophic factors (NTFs) compared to naïve undifferentiated conditions, and exhibited neuroprotective capacities both in culture and in a sciatic nerve injury model⁷.

Tissue engineering scaffolds provide a 3-D environment for cell attachment, growth and differentiation, maintain cell distribution and provide cell protection following transplantation⁸. We have shown that PLLA/PLGA scaffolds enhance NTF secretion by olfactory bulb cells⁹ and that fibrin/PLLA/PGLA scaffolds support cell proliferation, differentiation and organization¹⁰. Such scaffolds may also act as a reservoir for secreted NTFs, creating gradients capable of supporting morphogenesis and potentiation of their actions⁹. Based on these data, we hypothesized that an engineered composite tissue construct consisting of induced hOMSCs embedded in a fibrin matrix intermingled within a porous PLLA/PLGA scaffold, will act as a multi-effector device capable of supporting neurological recovery following complete spinal cord transection in the rat model.

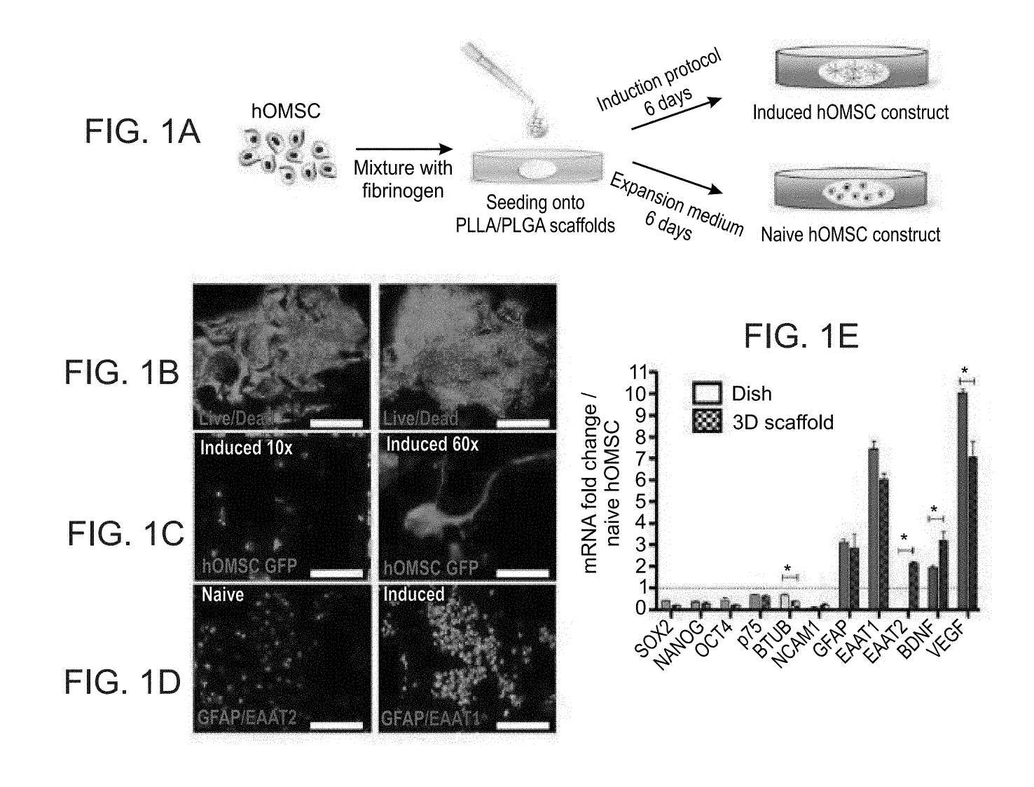

To test this hypothesis, we embedded hOMSCs within fibrinogen and thrombin that were cast on porous 3D PLLA/PLGA scaffolds. The cell-embedded scaffolds were then exposed to induction medium for 6 days, to yield induced-constructs (FIG. 1A). Control constructs were maintained in naïve growth medium (naïve-constructs). Prior to transplantation, cell viability within the construct was >95% (FIG. 1B). Confocal microscopic examination of induced-constructs embedded with GFP-labeled hOMSCs, revealed cells with elongated processes homogenously distributed within the constructs

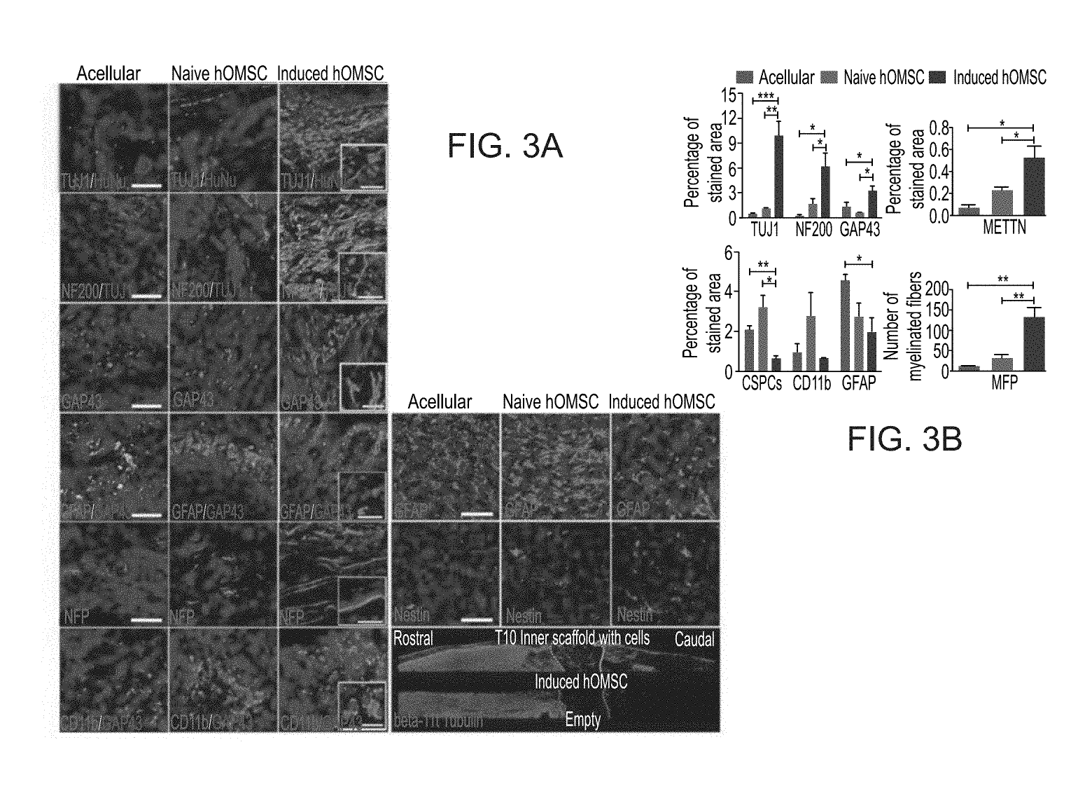

(FIG. 1C). In parallel, as we have previously observed^{6,8}, the induction process resulted in decreased expression of pluripotency, neural crest and neuronal-associated genes and in a substantial increase in the expression of astrocyte-related genes GFAP, EEAT1, EAAT2, BDNF and VEGF (FIG. 1D). Confocal immunofluorescence analyses confirmed the upregulation of GFAP and EAAT1 proteins in the induced-construct (FIG. 1E). To characterize the expected paracrine effects of the on-scaffold-induced cells, we collected conditioned media from naïve and induced hOMSC-embedded constructs to identify the secreted factors, using a human antibody array. Levels of secreted HGF, GDNF, BDNF, NT-3, VEGF, IGF-1/IGFBP3, ENA-78 and SCF, known to induce neuroprotection and/or regeneration, were significantly upregulated in induced-constructs compared to naïve-constructs. Furthermore, many of the identified factors, such as GM-CSF, LIF, SDF-1, IL-10, IL-6 and IL-4, shared both immune and trophic/neuroprotective/regenerative properties (FIG. 4).

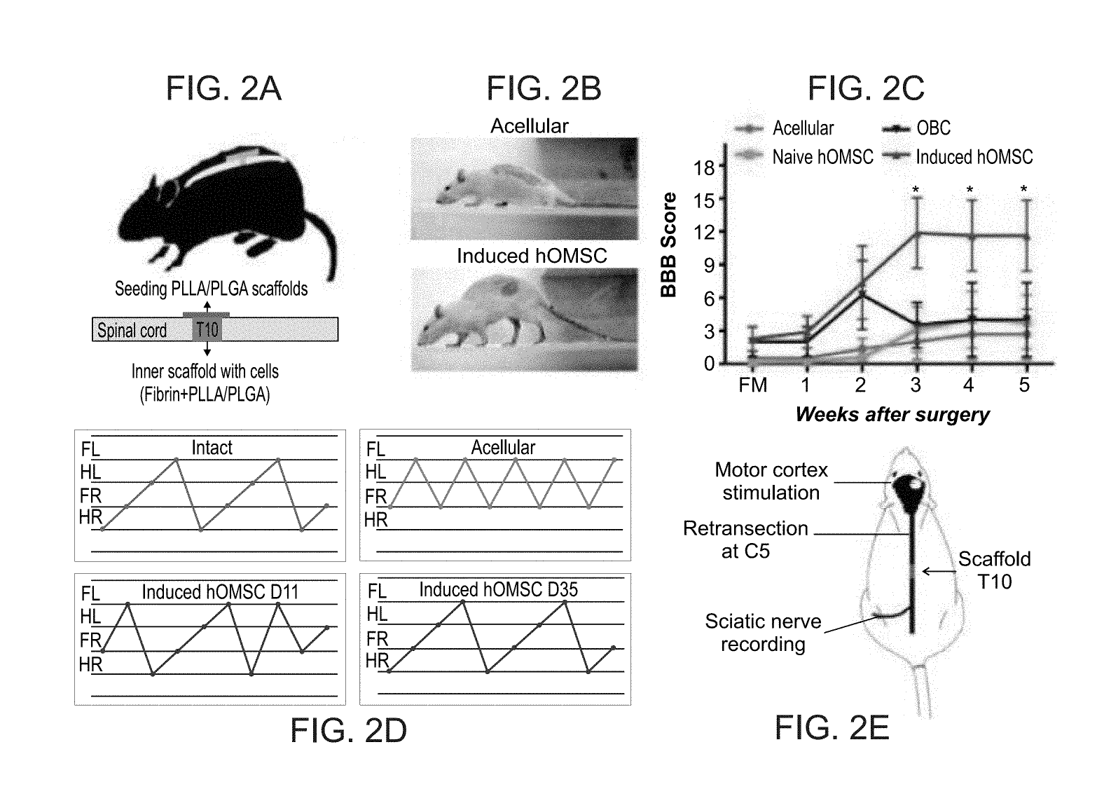

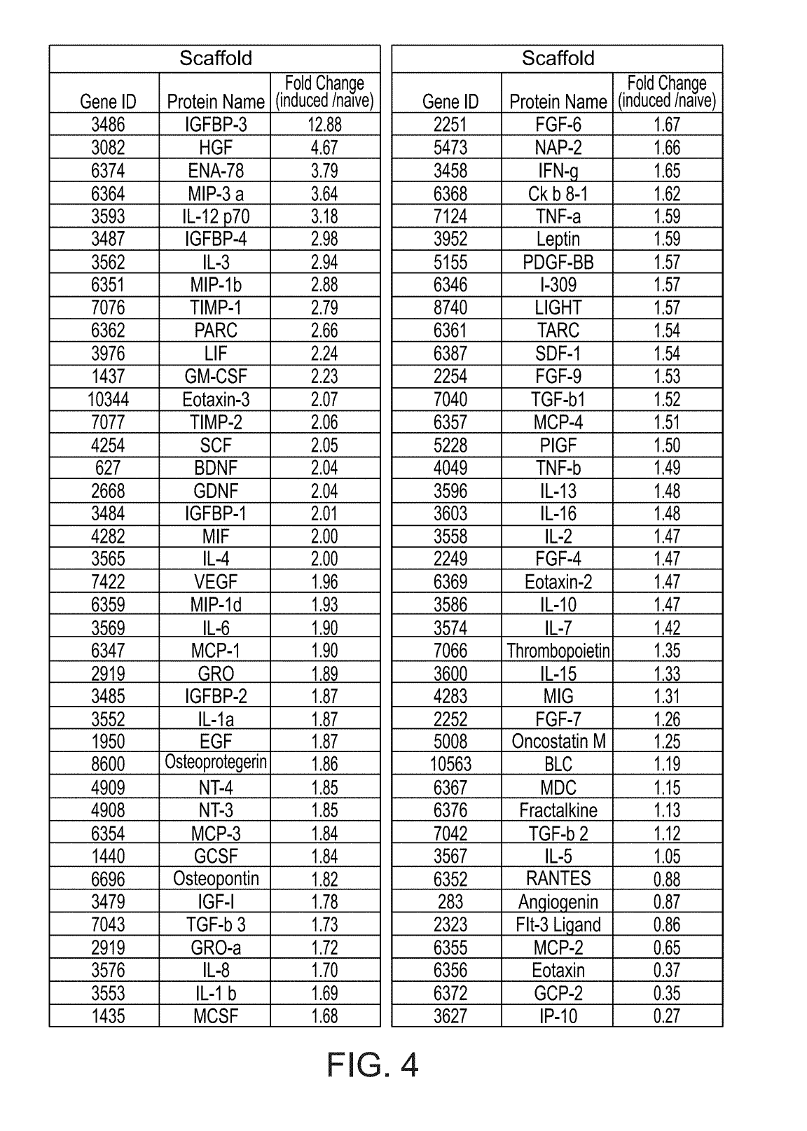

We then chose to assess the therapeutic potential of induced-hOMSC-constructs to support neurological recovery following complete transection of the rat spinal cord, a model that represents severe damage with minimal spontaneous recovery₁₁. Immediately following laminectomy and complete transection of the spinal cord at T10, hOMSC constructs (induced or naïve) or control acellular PLLA/PLGA scaffolds, fabricated to match the dimensions of the lesion, were implanted in the injured site (FIGS. 5A-B). Additional PLLA/PLGA scaffold was placed over the transected site and over the exposed rostral and caudal parts of the cord to provide structural support and minimize friction between spinal cord and the laminected bone (FIG. 2A).

Three weeks after injury, rats implanted with induced-constructs demonstrated higher motor and sensory recovery compared to rats implanted with naïve-constructs or with acellular scaffolds. Moreover, rats treated with induced-constructs demonstrated consistent weight support of the hind limbs (FIG. 2B), marked walking abilities, and an overall high recovery rate, with 63% exhibiting BBB¹² scores ≧17 (FIG. 2C). The high BBB scores are the compiled reflection of coordinated gait, plantar placement, weight support, recovery of toe clearance, trunk stability and predominant parallel paw and tail position, suggesting regained cortical motor control following treatment with the induced-constructs (FIG. 2D)₁₃. In sharp contrast, only 11% of animals treated with naïve-constructs had BBB scores ≧17, and none of the rats treated with acellular-constructs reached such scores. To further test the specificity of the observed results, an additional group of rats were implanted with constructs seeded with rat olfactory bulb-derived cells (OBC) that have been reported to support spinal cord injury³. As depicted in FIG. 2C, starting from 3 weeks post-implantation, the mean BBB score of rats treated with OBC-constructs was similar to that of animals treated with naïve-constructs, and was significantly lower than that of rats treated with induced-constructs.

To determine the electrophysiological basis of the observed motor recovery, motor cortexes of animals treated with either the induced-constructs or acellular scaffolds were stimulated with single spikes, and motor-evoked potentials were recorded from the isolated sciatic nerve at the hind limb level (FIG. 2E). Signal propagation from the motor cortex via cortico-spinal tracts, to the lower motor neurons was observed in the rats treated with induced-constructs, albeit less than in intact control animals (FIG. 2F). The amplitudes measured for induced-construct-treated animals were 3-fold higher than amplitudes observed in the acellular construct group, where they were barely detectable (FIG. 2G). The signal propagations in the induced-construct group were abolished by a second transection performed rostral to the first one at C5. Taken together, these results suggest partial restoration of the connectivity between the rostral and caudal segments of the spinal cord in animals treated with the induced-constructs, in line with previously reported observations^{2, 14}.

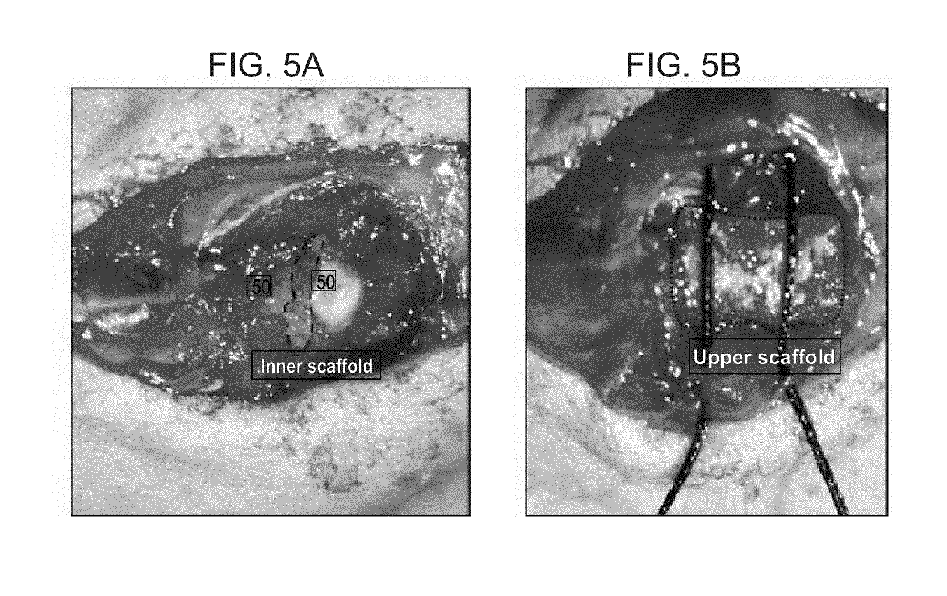

Complete spinal cord transection results in loss of all sensory functions caudal to the injured site. Here, we show that 75% of the rats treated with induced-constructs and assessed after 56 days for nociception, responded to nociceptive stimuli, while animals receiving the acellular construct failed to show any sensory response (FIG. 2H). MRI diffusion tensor imaging (MRI-DTI) was then performed on days 3 and 56 after surgery to understand the basis of the motor and sensory recovery (FIGS. 2J-K). Fiber tracking showed partial re-connection of fibers in the rats treated with the induced-constructs, while no reconnection was evidenced in the acellular scaffolds group (FIGS. 2I-J). Fractional anisotropy (FA) was calculated to characterize the directional properties of axonal bundles as a parameter for functional recovery following spinal injury₁₅. On day 56 of the study, FA values 0-4 mm caudal to the injury site were significantly higher among induced-construct rats compared to rats with acellular scaffolds, but lower than in intact rats, demonstrating improved directional organization of the axonal structure in the induced hOMSC group (FIG. 2K, FIG. 6).

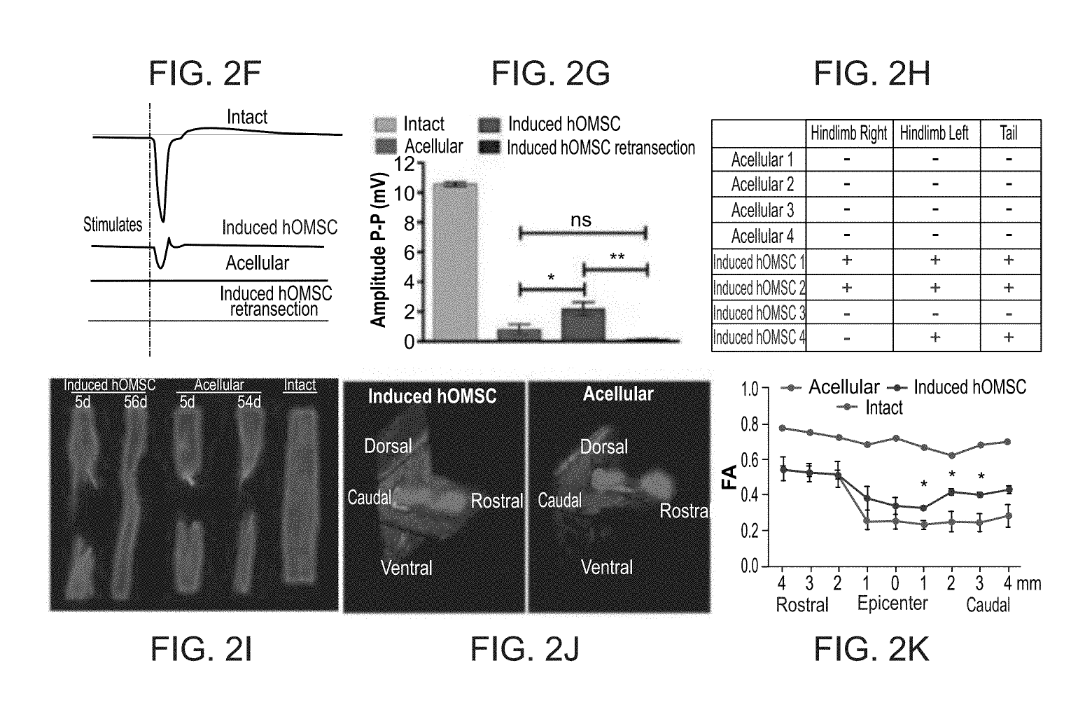

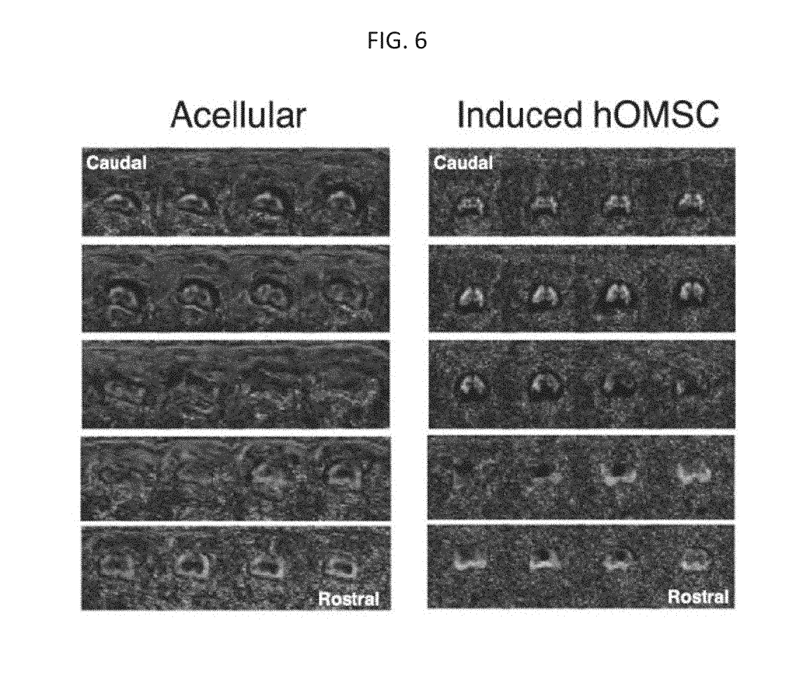

Next, to identify cellular processes potentially associated with recovery of the neuronal circuitry, we used histology and qualitative and quantitative immunofluorescence tools to examine the implantation site and its vicinity at the end of the experimental period (FIGS. 3A-B). While injury site cavitation was observed in animals implanted with acellular scaffolds, new tissue with organized a ventral white matter structure was identified in the induced-construct group (FIG. 7). The neuronal markers beta-III tubulin and neurofilament 200 were expressed in the induced hOMSC construct and caudally to them, but were barely identified in the acellular scaffolds or in naïve-constructs (FIG. 3A). Similar expression profiles were obtained for the axonal elongation marker GAP43 and the myelin basic protein (FIG. 3A). At the same time, induced-constructs displayed the lowest levels of the glial scar marker chondroitin sulfate proteoglycan (CSGP) and of its co-localization with the axonal elongation factor GAP43 (FIG. 3A). Astrocyte marker GFAP levels were also reduced in this group.

The cellular components and microenvironment of glial scars have been shown to inhibit axonal regeneration and re-establishment of neuronal circuitry¹. Our data indicate reduced glial scar formation at sites treated with the induced-construct, providing an explanation for the regenerative processes and consequential neurological recovery.

The neuroprogenitor marker nestin was most abundantly expressed in animals treated with the induced-constructs, while its expression was 50% and 75% lower in the naive-construct and acellular scaffold-treated animals, respectively (FIG. 3B).

Since nestin-positive progenitors can differentiate into either glia or neuronal cells at sites of spinal cord injury¹⁶, our data suggest that the induced-construct supports neural precursor proliferation.

Cells expressing CD11b, a marker of microglia activation within the CNS, tend to cluster at sites of injury and neurodegeneration. Here, we observed the highest level of CD11b expression at the sites treated with the control constructs, particularly in the vicinity of naïve hOMSC constructs, suggesting an inflammatory response to the inflicted injury and to the transplanted cells (FIG. 3A). In contrast, the lowest level of CD11b expression was observed at sites treated with the induced-constructs, indicating relatively low inflammatory responses at these sites. Taken together, the induced hOMSCs in the experimental constructs modulate the microglial response in a manner that favors reduced glia scar formation and possibly supports neuronal recovery (FIG. 3B).

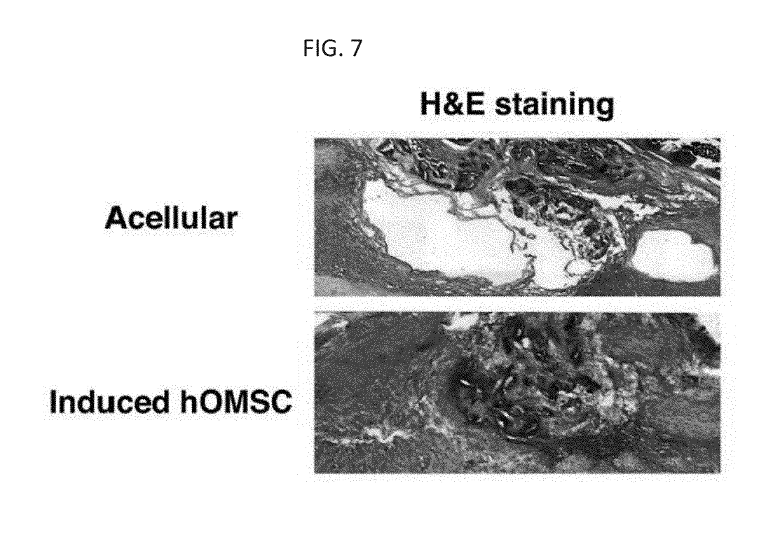

To determine whether the induced hOMSCs migrate from the constructs into the proximal and distal spinal cord, constructs engineered with GFP-labeled cells were prepared and implanted as described. A number of labeled cells were identified at day 28 after surgery at a distance of up to 4 mm both rostral and caudal to the implantation site (FIGS. 8A-B). However, the majority of cells were retained at the implantation site, suggesting that the effect of the experimental constructs was mainly mediated by neurotrophic and immunomodulatory factors locally secreted by induced hOMSCs.

It has been demonstrated that NTFs plays a major role in post-SCI recovery, by promoting cell survival, axonal growth, and even enabling axons to elongate and avoid the axon-inhibitory molecules of the glial scar¹⁷. We found that the induction protocol implemented here brought about increased secretion of a number of NTFs and immunomodulatory cytokines (FIG. 4) that have been shown to support axonal growth by counteracting the inhibitory microenvironment of the glial scar.

The most relevant NTFs for SCI repair are BDNF, NT-3/4, GDNF, VEGF, HGF and SDF-1¹⁸, which were all secreted by induced-constructs. BDNF- or NT-3-impregnated scaffolds have been reported to enhance formation of NF200-positive axons, neurite growth into scaffolds and reduce inflammatory responses, glial reactivity and CSPG expression at the interface between the scaffold and host spinal cord^{19, 20}. BDNF and NT-4 also enhance growth and regeneration of both descending rubrospinal and reticulospinal axonal networks that regulate spinal cord motor neural activities, also via GAP-43-positive axons²¹⁻²³. VEGF and NT-3 were both demonstrated to impart regenerative effects on cortico-spinal tracts^{24, 25}. In parallel, the glial scar can also be modulated by NTFs. HGF, secreted by induced hOMSCs, may play a dual role: inhibition of astrocyte-derived CSPGs, leading to increased axonal growth, and preservation of corticospinal tracts²⁶, ²⁷. SDF-1, also secreted by the induced constructs, was demonstrated to promote axon outgrowth in the presence of myelin inhibitors and to attract endogenous nestin-positive neural precursor cells to the injury site²⁸. Considering the complexity of wound healing in spinal cord injuries, it is possible that the synergistic activity of the differentially secreted factors by induced-constructs versus naïve hOMSCs, elicited the functional changes observed in the experimental animals²⁹.

In summary, we demonstrated that transplantation of artificial tissue constructs secreting regeneration-stimulating trophic factors, form growth-permissive topography that promotes axonal growth across the injury site. Following treatment of a complete spinal cord resection, substantial recovery was achieved, enabling paraplegic rats to walk independently. While other studies investigating recovery after complete transection have shown substantial histological outcomes alongside axonal elongation following implantation of neural precursors², our treatment based on astrocyte-like cells demonstrated regained walking abilities, motor coordination, sensory processing and electrical conduction from the brain to the hindlimbs enabled by extensive regenerative processes. Our combined approach using tissue engineering and cell therapy, counteracted processes which are known to limit spontaneous functional and structural recovery following SCI³⁰. The use of accessible cells in combination with biocompatible materials, makes our approach compelling for translation to clinical stages.

Materials and Methods

Naïve hOMSC cell culture: hOMSCs were obtained from oral mucosa biopsies after obtaining signed informed consent and the approval of the Institutional Helsinki Committee at the Baruch Padeh Medical Center, Poria, Israel by Dr. Shareef Araidy and Dr. Sammy Pour. hOMSCs were isolated and cultured in expansion medium consisting of low-glucose Dulbecco's modified Eagle's medium supplemented with 100 μg/ml streptomycin, 100 U/ml penicillin, (Biological Industries, Beit-Haemek, Israel), 2 mM glutamine (Invitrogen, Carlsbad, Calif., USA) and 10% fetal calf serum (FCS) (Gibco), as described by Marynka-Kalmani et al. ⁶. Briefly, biopsies were incubated overnight at 4° C. in dispase (Sigma, Israel). Then, the epithelial layer was separated from the lamina propria and the latter was minced into 0.5 mm³ pieces and placed in 35 mm culture dishes (Nunc). Expansion medium was gently added to the explants to enhance their attachment to the floor of the dish. Cells that emigrated from the explant to the culture dishes were harvested with 0.25% trypsin (Biological Industries, Beit-Haemek, Israel) and seeded at a cell density of 4×10⁴ cells/cm². Cells were passaged at 70-80% confluence. All experiments used hOMSCs at passages 4-20.

hOMSC seeding and differentiation: Naïve hOMSCs were harvested with trypsin (Biological Industries, Israel), counted and aliquoted (5×10⁵ cells/tube). Cells were resuspended in 5 μl human thrombin (Omrix Biopharmaceuticals, Israel) and further mixed with 5 μl human fibrinogen solution (Biological Active Components 2, Omrix Biopharmaceuticals, Israel) and then immediately placed into the rigid PLLA/PLGA scaffold (50% PLLA and 50% PLGA) which had been fabricated utilizing a particulate leaching technique to achieve pore sizes of 212-600 μm and 93% porosity. Briefly, PLLA (Polysciences) and PLGA (Boehringer Ingelheim) were dissolved 1:1 in chloroform to yield a 5% (w/v) polymer solution; 0.24 ml of this solution was loaded into molds packed with 0.4 g sodium chloride particles. The solvent was allowed to evaporate overnight, and the sponges were subsequently immersed for 8 h in distilled water, which was changed every hour, to leach the salt and create an interconnected, porous structure. Final PLLA/PLGA sponges were circular with a diameter of 2 mm and thickness of 600 um. Before use, sponges were soaked overnight in 70% (v/v) ethyl alcohol and washed three times with PBS. After addition of the fibrin/thrombin cellular solution to the PLLA/PLGA scaffold, the construct was placed on 24-wells plates (non-tissue culture) and allowed to polymerize for 30 min inside the incubator (37° C., 5% CO_{2,} high humidity). hOMSC expansion medium (1 mL) was then added to each well, and scaffolds were cultured overnight. The next day, the medium was replaced; cells to be used in their naïve state were maintained for six days in growth medium, while the differentiated cells were maintained in differentiation media I and II for a total of six days, as described for hOMSC astrocyte induction⁷.

Real-time PCR: Total RNA from scaffolds (n=3) was isolated using the TRI reagent (Invitrogen, Carlsbad, Calif., USA), according to the supplier's recommendations. RNA (2 μg) was reverse transcribed with random primers and SuperScriptIII (Invitrogen, Carlsbad, Calif., USA). Real-time PCR of the genes of interest was performed in a StepOnePlus™ (Applied Biosystems), using PlatinumR SYBRR Green qPCR SuperMix UDG with ROX (Invitrogen, Carlsbad, Calif., USA). PCR amplification was performed over 40 cycles (program: 2 min at 50° C.; 2 min at 95° C.; 40 repeats of 15 s at 95° C. and 30 s at 60° C.). Data were quantified using the ΔΔCt method, and normalized to the lactate dehydrogenase A (LDHA) housekeeping gene. ΔCt of undifferentiated cultures served as baseline values. Data are presented as the mean ±standard error of the mean (SEM) change from the baseline. Primer sequences used for RT-PCR analysis are presented in the materials sections.

Cytokine Array: Cytokine levels in conditioned medium of naïve and induced-constructs were compared using the human RayBio® G-Series Cytokine Array (RayBiotech, Inc, USA), as per the manufacturer's guidelines. Total cell protein served as the normalization factor between conditions. Naïve hOMSCs served as reference and results were expressed as fold-change from naïve conditions per milligram of protein.

Tissue Immunofluorescence

Spinal cord analysis—Rats were sacrificed with CO₂ and immediately perfused with PFA 4%. Spinal cords were dissected, embedded in OCT and sectioned (20 μm) using a cryostat (Leica CM1850, Germany). Sections were blocked in 5% goat serum, 1% BSA, and 0.05% Triton-X in PBS for 2 hr and then incubated with primary antibodies overnight at 4° C. For immunofluorescence, sections were incubated with dye-conjugated secondary antibodies. For immunohistochemistry hematoxylin and eosin staining was performed. Sections from the same rats were used for immunochemistry and immunofluorescence. Histological staining was performed on 3-11 sections of each spinal cord. Using custom-made MATLAB software, the region of interest (ROI) was manually identified to exclude the non-scaffold area. The resultant image was decomposed to blue, green and red channels. For each channel, a threshold filter was applied at 35% of the maximum intensity value to remove noise. The total pixels area was calculated and normalized to the actual area of the ROI.

MATLAB scripts were programmed to automatically count elongated elements representing axons in Myelin basic protein (MBP) immunofluorescence images. Images were cleaned using morphological operators. The resultant binary image was segmented by selecting connected areas. Areas larger than a certain threshold were automatically excluded from the ROI to avoid miscalculation of large bundles of connected neurons. For each region, second-order moments were calculated to obtain major and minor axis lengths. All areas containing a major to minor axis ratio >5 were identified as elongated axons. The number of elongated axons in each image was counted.

Spinal cord injury and construct implantation: All animal experiments were performed in strict compliance with protocols approved by Technion/TAU Ethics Committees. Adult female Sprague-Dawley rats were anesthetized with a mixture of xylazine (100-150 mg/kg) and ketamine (60-90 mg/kg) and maintained with isofluorane (Harvard Apparatus, USA) during surgery. After laminectomy at the 9th-11th thoracic vertebral levels the spinal cord was completely transected at the T10 level, using a microscissor (Kent Scientific, USA). The rostral and caudal stumps were lifted to ensure complete transection and a hook (Kent Scientific, USA) was passed circularly inside the generated gap to confirm that no fibers remained at the bottom part of the spine canal. Then, the constructs (2 mm×2 mm×0.6 mm) (acellular or hOMSC-embedded scaffolds) were inserted precisely between both caudal and rostral parts of the spinal cord and sealed with an acellular PLLA/PLGA scaffold, which provided structural support. Muscle layers and skin were sutured and after surgery, the rats were placed in temperature- controlled incubation chambers until they awoke. They were then transferred to cages, and bladder evacuation was applied two times each day, until regain of bladder function. Antibiotics (cephalexin, 10 mg/kg body weight) were injected into the rats daily for one week. Buprenorphine (Bayer) was administered at a dose of 0.01-0.05 mg/kg before surgery and three days after. Cyclosporin (10 mg/kg/d) (Novartis) was administered daily to all rats one day before surgery through 5 days post-surgery.

MRI-DTI

MRI protocol: MRI was performed, with the assistance of Bioimage Ltd., in a 7T MRI system (Bruker, Germany), using a 20 mm surface coil placed on the back of the rats, at the injury site. Rats were anesthetized using 1-3% isoflurane and maintained at 37°; breathing was monitored with a respiratory sensor. The MRI protocol included the following sequences:

T2 RARE: Sagittal T2-weighted imaging was performed in order to localize the axial slices in the correct location, including upstream and downstream regions adjacent to the injury site. T2 RARE included the following parameters: TR/TE=1200/16, RARE factor=4, no. of averages=4, 20 slices of 0.8 mm, in-plane resolution of 0.17×0.2 mm (matrix size 128×128 and FOV of 25.6×22.8 mm).

DTI: DTI was performed under the following conditions: TR/TE=4500/30 ms, 4 EPI segments, Δ/δ=10/4.5 ms, 15 non-collinear gradient directions with a single b value shell at 1000 sec/mm2 and one image with b value of 0 sec/mm2 (referred to as b0), 3 averages, 2 repetitions. Geometrical parameters were: 18 slices of 1 mm thickness (brain volume) and in-plane resolution of 0.156×0.156 mm² (matrix size of 128×128 and FOV of 20 mm²). The duration of each DTI repetition was 14:24 min.

DTI fiber tracking: DTI calculation and fiber tracking were performed using the ExploreDTI software (Leemans et al., 2009). The tensors obtained were spectrally decomposed to their eigen-components. The eigen-values were used to calculate FA and MD maps. Tractography was applied using Deterministic (streamline) fiber tracking, terminating at voxels with FA lower than 0.3 or following a tract orientation change higher than 30° (Basser et al. 2000). Fibers that passed through a manually selected region of interest (ROI) were plotted. The fibers were plotted as streamlines. The masks obtained were overlaid over the color-coded FA image.

Motor analysis in spinal cord injury: Rats were subjected to BBB and gait analysis assays. The assays were performed on a setup enabling simultaneous photography of the sagittal and coronal planes. Measurements were made 1-4 d following implantation of a construct, followed by measurements every 7 days, up until 56 d after implantation. All measurements were made at the same time of day to avoid circadian variability. Two experimenters blinded to the treatment, performed the test according to the BBB method ¹². Baseline BBB was determined from the first test after surgery. The weekly score was the maximum score obtained during each calendar week. Animals that died in surgery (or within 72 hours of surgery) were excluded from the experiment. BBB scores of animals that died after this period were defined as 0. In one case, a missing weekly score was extrapolated using zero order hold.

Electrophysiology: Following ketamine/xylazine anesthesia, rats were fixed into the stereotaxic apparatus and a midline incision was made in the head skin. The cranium was exposed and two screw electrodes for electrical stimulation were implanted 2 mm to the right of the midline, at −1.0 mm and +4.0 mm anterior and posterior to the bregma, respectively. The screw electrodes were connected to the output terminals of the SD9 stimulator (Grass Technologies, Warwick, R.I.). The sciatic nerve at the rear of the left leg was exposed and two hook silver wire electrodes were inserted. Another wire was inserted into the footpad of the leg and served as a ground electrode. The hook electrodes were connected to the unity gain headstage built on a dual TL072 operational amplifier (Texas Instruments) and powered from two 9V batteries. The amplified signals were band-pass filtered between 0.1 Hz and 3 kHz (7P511 AC wideband preamplifier with 7DA driver amplifier, Grass Technologies, Warwick, R.I.), digitized (NI USB-6341 analog-to-digital converter, National Instruments), acquired at 10 kHz and stored on a personal computer running WinWCP software package (courtesy of Dr. John Dempster, University of Strathclyde, UK). The stimulation intensity was chosen according to the hindlimb contraction and appearance of the reliable sciatic nerve compound action potential (CAP) in the first animal, and maintained throughout the experiment. Amplitudes were measured maximal peak-to-peak.

Sensory examination: Sensory evaluation was performed at the end of the experimental period (56 days after surgery), using the pinch technique. The nociceptive stimulus was applied in both hindlimbs and tail. Responses were considered binary (responsive or nonresponsive, scored as positive or negative, respectively). The responsiveness criterion was defined as a deep-brain response, manifested by a vocal cue, head turn or a withdrawal effect of the evaluated hindlimbs or tail, generated at the pinched site.

Statistical analysis: Results are expressed as mean ±SEM. All analyses were performed using MATLAB/ Prism. Graphs were generated by Prism 5 software (USA). Differences between two groups were statistically analyzed by a T test, while one-way ANOVA was applied to compare between three groups and Newman-Keuls multiple comparison posthoc test was used to characterize specific differences between groups. For the cell transplantation in vivo experiment, two-way ANOVA with Bonferroni posthoc test was performed. Significance levels: *p<0.05, **p<0.01, ***p<0.001.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Cregg, J. M. et al. Functional regeneration beyond the glial scar. Exp Neurol 253, 197-207 (2014).

2. Lu, P. et al. Long-distance growth and connectivity of neural stem cells after severe spinal cord injury. Cell 150, 1264-1273 (2012).

3. Ramon-Cueto, A., Cordero, M. I., Santos-Benito, F. F. & Avila, J. Functional recovery of paraplegic rats and motor axon regeneration in their spinal cords by olfactory ensheathing glia. Neuron 25, 425-435 (2000).

4. Coumans, J. V. et al. Axonal regeneration and functional recovery after complete spinal cord transection in rats by delayed treatment with transplants and neurotrophins. J Neurosci 21, 9334-9344 (2001).

5. Tabakow, P. et al. Functional regeneration of supraspinal connections in a patient with transected spinal cord following transplantation of bulbar olfactory ensheathing cells with peripheral nerve bridging. Cell Transplant (2014).

6. Marynka-Kalmani, K. et al. The lamina propria of adult human oral mucosa harbors a novel stem cell population. Stem Cells 28, 984-995 (2010).

7. Ganz, J. et al. Astrocyte-like cells derived from human oral mucosa stem cells provide neuroprotection in vitro and in vivo. Stem cells translational medicine 3, 375-386 (2014).

8. Levenberg, S. & Langer, R. Advances in tissue engineering. Curr Top Dev Biol 61, 113-134 (2004).

9. Blumenthal, J., Cohen-Matsliah, S. I. & Levenberg, S. Olfactory bulb-derived cells seeded on 3D scaffolds exhibit neurotrophic factor expression and pro-angiogenic properties. Tissue Eng Part A 19, 2284-2291 (2013).

10. Shandalov, Y. et al. An engineered muscle flap for reconstruction of large soft tissue defects. Proc Natl Acad Sci U S A 111, 6010-6015 (2014).

11. Talac, R. et al. Animal models of spinal cord injury for evaluation of tissue engineering treatment strategies. Biomaterials 25, 1505-1510 (2004).

12. Basso, D. M., Beattie, M. S. & Bresnahan, J. C. A sensitive and reliable locomotor rating scale for open field testing in rats. J Neurotrauma 12, 1-21 (1995).

13. Li, Y., Alam, M., Guo, S., Ting, K. H. & He, J. Electronic bypass of spinal lesions: activation of lower motor neurons directly driven by cortical neural signals. Journal of neuroengineering and rehabilitation 11, 107 (2014).

14. Garcia-Alias, G. et al. Differential motor and electrophysiological outcome in rats with mid-thoracic or high lumbar incomplete spinal cord injuries. Brain Res 1108, 195-204 (2006).

15. Kelley, B. J. et al. Diffusion tensor imaging as a predictor of locomotor function after experimental spinal cord injury and recovery. J Neurotrauma 31, 1362-1373 (2014).

16. Matsumura, S. et al. Characterization of nestin expression in the spinal cord of GFP transgenic mice after peripheral nerve injury. Neuroscience 170, 942-953 (2010).

17. Teng, Y. D. et al. Functional multipotency of stem cells: a conceptual review of neurotrophic factor-based evidence and its role in translational research. Current neuropharmacology 9, 574-585 (2011).

18. Silva, N. A., Sousa, N., Reis, R. L. & Salgado, A. J. From basics to clinical: a comprehensive review on spinal cord injury. Prog Neurobiol 114, 25-57 (2014).

19. Jain, A., Kim, Y. T., McKeon, R. J. & Bellamkonda, R. V. In situ gelling hydrogels for conformal repair of spinal cord defects, and local delivery of BDNF after spinal cord injury. Biomaterials 27, 497-504 (2006).

20. Taylor, S. J., McDonald, J. W., 3rd & Sakiyama-Elbert, S. E. Controlled release of neurotrophin-3 from fibrin gels for spinal cord injury. Journal of controlled release: official journal of the Controlled Release Society 98, 281-294 (2004).

21. Kobayashi, N. R. et al. BDNF and NT-4/5 prevent atrophy of rat rubrospinal neurons after cervical axotomy, stimulate GAP-43 and Talphal-tubulin mRNA expression, and promote axonal regeneration. J Neurosci 17, 9583-9595 (1997).

22. Liu, Y. et al. Transplants of fibroblasts genetically modified to express BDNF promote regeneration of adult rat rubrospinal axons and recovery of forelimb function. J Neurosci 19, 4370-4387 (1999).

23. Jin, Y., Fischer, I., Tessler, A. & Houle, J. D. Transplants of fibroblasts genetically modified to express BDNF promote axonal regeneration from supraspinal neurons following chronic spinal cord injury. Exp Neurol 177, 265-275 (2002).

24. Tuszynski, M. H. et al. NT-3 gene delivery elicits growth of chronically injured corticospinal axons and modestly improves functional deficits after chronic scar resection. Exp Neurol 181, 47-56 (2003).

25. Facchiano, F. et al. Promotion of regeneration of corticospinal tract axons in rats with recombinant vascular endothelial growth factor alone and combined with adenovirus coding for this factor. J Neurosurg 97, 161-168 (2002).

26. Jeong, S. R. et al. Hepatocyte growth factor reduces astrocytic scar formation and promotes axonal growth beyond glial scars after spinal cord injury. Exp Neurol 233, 312-322 (2012).

27. Kitamura, K. et al. Human hepatocyte growth factor promotes functional recovery in primates after spinal cord injury. PLoS One 6, e27706 (2011).

28. Jaerve, A., Bosse, F. & Muller, H. W. SDF-1/CXCL12: its role in spinal cord injury. Int J Biochem Cell Biol 44, 452-456 (2012).

29. Tetzlaff, W. et al. A systematic review of cellular transplantation therapies for spinal cord injury. J Neurotrauma 28, 1611-1682 (2011).

30. Jones, L. L., Oudega, M., Bunge, M. B. & Tuszynski, M. H. Neurotrophic factors, cellular bridges and gene therapy for spinal cord injury. J Physiol 533, 83-89 (2001).

1. A method of treating a spinal cord injury in a subject in need thereof comprising implanting a scaffold into the spinal cord of a subject, wherein the scaffold is seeded with oral mucosa stem cells (OMSC) and/or cells that have been ex vivo differentiated from said OMSCs, thereby treating the spinal cord injury.

2. A scaffold comprising oral mucosa stem cells (OMSC) and/or cells that have been ex vivo differentiated from said OMSCs.

3. (canceled)

4. The method of claim 1, wherein said implanting is effected at the spinal cord.

5. (canceled)

6. The method of claim 1, wherein said cells secrete at least one neurotrophic factor.

7. The method, of claim 1, wherein said neurotrophic factor is selected from the group consisting of: glial-derived neurotrophic factor (GDNF), brain-derived neurotrophic factor (BDNF), neurotrophin-3 (NT-3), neurotrotrophin-4/5; Neurturin (NTN), Neurotrophin-4, Persephin, artemin (ART), ciliary neurotrophic factor (CNTF), insulin growth factor-I (IGF-I) and Neublastin.

8-10. (canceled)

11. The method, of claim 1, wherein said scaffold comprises a therapeutic agent.

12-15. (canceled)

16. The method, of claim 1, wherein said scaffold is fabricated from a biodegradable porous material.

17. (canceled)

18. The method, of claim 1, wherein said scaffold is fabricated from a non-synthetic material.

19. The method, of claim 1, wherein said scaffold is fabricated from a material selected from the group consisting of poly(L-lactic acid), poly(lactic acid-co-glycolic acid), collagen-GAG, collagen, fibrin, poly(anhydride), poly(hydroxy acid), poly(ortho ester), poly(propylfumerate), poly(caprolactone), polyamide, polyamino acid, polyacetal, biodegradable polycyanoacrylate, biodegradable polyurethane and polysaccharide, polypyrrole, polyaniline, polythiophene, polystyrene, polyester, non-biodegradable polyurethane, polyurea, poly(ethylene vinyl acetate), polypropylene, polymethacrylate, polyethylene, polycarbonate and poly(ethylene oxide).

20. The method, of claim 1, wherein said scaffold is fabricated from a material comprising poly(L-lactic acid) and poly(lactic acid-co-glycolic acid).

21. The method of claim 4, wherein said scaffold comprises a protruding scaffold and a supporting scaffold, wherein at least a portion of said protruding scaffold is inserted into a lesioned area of the spinal cord so as to contact an injury or diseased site, wherein said supporting scaffold does not protrude into said injury or diseased site and is in contact with the rostral and/or caudal dura of the spinal cord, wherein said supporting scaffold and said protruding scaffold are in physical contact with one another following said implanting and said supporting scaffold is orientated with respect to said protruding scaffold to form a shape comprising a T following said implanting.

22. The method of claim 21, wherein said protruding scaffold and said supporting scaffold are part of a single element.

23. The method of claim 22, wherein said protruding scaffold is a separate element to said supporting scaffold.

24. The method of claim 23, wherein said protruding scaffold is implanted prior to said supporting scaffold.

25. The method of claim 21, wherein said protruding scaffold is carved into a shape of said lesioned area of the spinal cord.

26. The scaffold of claim 2, wherein the scaffold is shaped in a T shape.

27. The scaffold of claim 2, being of dimensions such that it can protrude into a spinal cord lesion.

28. The method of claim 1, wherein said cells that have been ex vivo differentiated from said OMSCs are ex vivo differentiated prior to seeding said scaffold.

29. The method of of claim 1, wherein said cells that have been ex vivo differentiated from said OMSCs are ex vivo differentiated following seeding said scaffold.

30. The scaffold of claim 2, wherein said cells secrete at least one neurotrophic factor.

31. The scaffold of claim 2, wherein said neurotrophic factor is selected from the group consisting of: glial-derived neurotrophic factor (GDNF), brain-derived neurotrophic factor (BDNF), neurotrophin-3 (NT-3), neurotrotrophin-4/5; Neurturin (NTN), Neurotrophin-4, Persephin, artemin (ART), ciliary neurotrophic factor (CNTF), insulin growth factor-I (IGF-I) and Neublastin.

32. The scaffold of claim 2, being fabricated from a biodegradable porous material.

33. The scaffold of claim 2, being fabricated from a material selected from the group consisting of poly(L-lactic acid), poly(lactic acid-co-glycolic acid), collagen-GAG, collagen, fibrin, poly(anhydride), poly(hydroxy acid), poly(ortho ester), poly(propylfumerate), poly(caprolactone), polyamide, polyamino acid, polyacetal, biodegradable polycyanoacrylate, biodegradable polyurethane and polysaccharide, polypyrrole, polyaniline, polythiophene, polystyrene, polyester, non-biodegradable polyurethane, polyurea, poly(ethylene vinyl acetate), polypropylene, polymethacrylate, polyethylene, polycarbonate and poly(ethylene oxide).
